(19) United States
(12) Patent Application Publication
(10) Pub. No.: US 2023/0170044 A1
(43) Pub. Date: Jun. 1, 2023

(54) SYSTEM AND METHOD FOR SCREENING PHENOTYPIC TARGETS ASSOCIATED WITH A DISEASE USING IN-SILICO TECHNIQUES

(71) Applicant: Innoplexus AG, Eschborn (DE)

(72) Inventors: Om Sharma, Pune (IN); Irfan Yunus Tamboli, Pune (IN); Uma Chandran, Thiruvananthapuram (IN)

(73) Assignee: Innoplexus AG, Eschborn (DE)

(21) Appl. No.: 17/539,744

(22) Filed: Dec. 1, 2021

(51) Int. Cl. G16B 20/20; G16B 20/10; G16B 30/00; C12Q 1/6809

(52) U.S. Cl. CPC G16B 20/20; G16B 20/10; G16B 30/00; C12Q 1/6809

(57) ABSTRACT

A system for screening phenotypic targets associated with a disease using in-silico techniques. The system communicably coupled to a phenotype ontological databank including a plurality of phenotypes and phenotypic targets associated with each of the plurality of phenotypes; wherein the system includes a processor communicably coupled to a memory. The processor configured to receive a first input of the disease, receive a second input relating to at least one phenotype associated with the disease, identify for each of the at least one phenotype a plurality of similar phenotypes relating to a particular phenotype of the at least one phenotype of the second input, determine a similarity score for each of the plurality of similar phenotypes in comparison with the particular phenotype of the at least one phenotype of the second input, extract, from the phenotype ontological databank, phenotypic targets associated with similar phenotypes having similarity score higher than a first predefined threshold, compute a cumulative score of the phenotypic targets based on a plurality of parameters, wherein the cumulative score of a given phenotypic target is indicative of relevance thereof with respect to the disease, screen out phenotypic targets with cumulative score lower than a second predefined threshold, compute relevant pathways for the phenotypic targets by performing Highly dysregulated pathway analysis (HDPA) for the screened phenotypic targets, compute mechanistic factors attributing to regulation of similar phenotypes and pathological information of the disease in association with the screened phenotypic targets.

### TECHNICAL FIELD

The present disclosure relates, generally, to screening techniques based on phenotypes. More specifically, the present disclosure relates to a system and a method for screening phenotypic targets associated with a disease using in-silico techniques.

### BACKGROUND

The process of drug discovery in the pharma industry is usually done using two different approaches i.e., Target Drug Discovery (TDD) and Phenotypic Drug Discovery (PDD), but with recurring setbacks and failures in the clinical trials of the investigation drugs being developed using the target-based approach (TDD), the pharma industry is now leaning more towards phenotypic-based approach (PDD). Most of the existing target-based approaches primarily focuses on identifying a target protein that can be switched on and off to gain therapeutic benefits over a disease. However, these approaches often ignore the role of phenotypes that may be responsible for driving a disease pathology.

The existing in-vitro approaches that are based on studying the role of multiple phenotypes in identifying targets is highly time consuming and costly. Such approaches might miss out on important targets that are responsible for driving different phenotypes but have not been yet identified to associate with disease pathology. Target identification platforms that consider the contribution of multiple phenotypes to develop a suitable intervention approach is the need of hour.

Therefore, in the light of the foregoing discussion, there still exists a need to overcome the aforementioned drawbacks associated with known techniques for screening of phenotypes and phenotypic targets associated to a disease.

### SUMMARY

An object of the present disclosure is to provide a system and a method for screening phenotypic targets associated with a disease using in-silico techniques. Another object of the present disclosure is to provide a solution that overcomes at least partially the problems encountered in the prior art.

In one aspect, an embodiment of the present disclosure provides a system for screening phenotypic targets associated with a disease using in-silico techniques, the system communicably coupled to
a phenotype ontological databank comprising information pertaining to a plurality of phenotypes and phenotypic targets associated with each of the plurality of phenotypes;
   wherein the system comprises a processor communicably coupled to a memory, and wherein the processor is configured to execute machine readable instructions that cause the system to perform the following operation:
receive a name of the disease as a first input;
receive at least one phenotype associated with the disease as a second input;
identify for each of the at least one phenotype a plurality of similar phenotypes relating to the particular phenotype of the at least one phenotype of the second input;
determine a similarity score for each of the plurality of similar phenotypes in comparison with the particular phenotype of the at least one phenotype of the second input;
extract, from the phenotype ontological databank, phenotypic targets associated with similar phenotypes having similarity score higher than a first predefined threshold;
compute a cumulative score of the phenotypic targets based on a plurality of parameters, wherein the cumulative score of a given phenotypic target is indicative of relevance thereof with respect to the disease;
screen out phenotypic targets with cumulative score lower than a second predefined threshold;
compute relevant pathways for the phenotypic targets by performing Highly dysregulated pathway analysis (HDPA) for screened phenotypic targets;
compute mechanistic factors attributing to regulation of similar phenotypes and pathological information of the disease in association with the screened phenotypic targets.

In another aspect, an embodiment of the present disclosure provides a method for screening phenotypic targets associated with a disease using in-silico techniques, wherein the method is implemented using a system communicably coupled to a phenotype ontological databank comprising information pertaining to a plurality of phenotypes and phenotypic targets associated with each of the plurality of phenotypes, wherein the method comprises
receiving a name of the disease as a first input;
receiving at least one phenotype associated with the disease as a second input;
identifying for each of the at least one phenotype a plurality of similar phenotypes relating to the particular phenotype of the at least one phenotype of the second input;
determining a similarity score for each of the plurality of similar phenotypes in comparison with the particular phenotype of the at least one phenotype of the second input;
extracting, from the phenotype ontological databank, phenotypic targets associated with similar phenotypes having similarity score higher than a first predefined threshold;
computing a cumulative score of the phenotypic targets based on a plurality of parameters, wherein the cumulative score of a given phenotypic target is indicative of relevance thereof with respect to the disease;
screen out phenotypic targets with cumulative score lower than a second predefined threshold;
computing relevant pathways for the phenotypic targets by performing Highly dysregulated pathway analysis (HDPA) for the screened phenotypic targets;
computing mechanistic factors attributing to regulation of similar phenotypes and pathological information of the disease in association with the screened phenotypic targets.

Additional aspects, advantages, features and objects of the present disclosure will be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a block diagram of a system for screening phenotypic targets associated with a disease using in-silico techniques, in accordance with an embodiment of the present disclosure;

FIG. 2 is a system for identifying a plurality of similar phenotypes and extracting phenotypic targets associated with similar phenotypes having similarity score higher than a first predefined threshold, in accordance with implementation of the present implementation;

FIG. 3 is a graph to prioritize the screened phenotypic targets based on the cumulative score, in accordance with implementation of the present disclosure;

FIG. 4 is a Pathway-Target-Phenotype (PTP) network, in accordance with the embodiments of the present disclosure;

FIGS. 5A and 5B collectively illustrate a flowchart depicting steps of a method for screening phenotypic targets associated with a disease using in-silico techniques, in accordance with the embodiments of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, an embodiment of the present disclosure provides a system for screening phenotypic targets associated with a disease using in-silico techniques, the system communicably coupled to
a phenotype ontological databank comprising information pertaining to a plurality of phenotypes and phenotypic targets associated with each of the plurality of phenotypes;
   wherein the system comprises a processor communicably coupled to a memory, and wherein the processor is configured to execute machine readable instructions that cause the system to perform the following operation:
receive a name of the disease as a first input;
receive at least one phenotype associated with the disease as a second input;
identify for each of the at least one phenotype a plurality of similar phenotypes relating to the particular phenotype of the at least one phenotype of the second input;
determine a similarity score for each of the plurality of similar phenotypes in comparison with the particular phenotype of the at least one phenotype of the second input;
extract, from the phenotype ontological databank, phenotypic targets associated with similar phenotypes having similarity score higher than a first predefined threshold;
compute a cumulative score of the phenotypic targets based on a plurality of parameters, wherein the cumulative score of a given phenotypic target is indicative of relevance thereof with respect to the disease;
screen out phenotypic targets with cumulative score lower than a second predefined threshold;
compute relevant pathways for the phenotypic targets by performing Highly dysregulated pathway analysis (HDPA) for screened phenotypic targets;
compute mechanistic factors attributing to regulation of similar phenotypes and pathological information of the disease in association with the screened phenotypic targets.

In another aspect, an embodiment of the present disclosure provides a method for screening phenotypic targets associated with a disease using in-silico techniques, wherein the method is implemented using a system communicably coupled to a phenotype ontological databank comprising information pertaining to a plurality of phenotypes and phenotypic targets associated with each of the plurality of phenotypes, wherein the method comprises
receiving a name of the disease as a first input;
receiving at least one phenotype associated with the disease as a second input;
identifying for each of the at least one phenotype a plurality of similar phenotypes relating to the particular phenotype of the at least one phenotype of the second input;
determining a similarity score for each of the plurality of similar phenotypes in comparison with the particular phenotype of the at least one phenotype of the second input;
extracting, from the phenotype ontological databank, phenotypic targets associated with similar phenotypes having similarity score higher than a first predefined threshold;
computing a cumulative score of the phenotypic targets based on a plurality of parameters, wherein the cumulative score of a given phenotypic target is indicative of relevance thereof with respect to the disease;
screen out phenotypic targets with cumulative score lower than a second predefined threshold;
computing relevant pathways for the phenotypic targets by performing Highly dysregulated pathway analysis (HDPA) for the screened phenotypic targets;
computing mechanistic factors attributing to regulation of similar phenotypes and pathological information of the disease in association with the screened phenotypic targets.

In one aspect, the present disclosure seeks to provide a system for screening phenotypic targets associated with a disease using in-silico techniques. Herein, the term “disease” refers to an abnormality that results in negatively affecting structure or functioning of a part or a whole of an organism. Herein, the phenotypes refer to a set of observable characteristics or traits of the organism. The term “phenotype” covers physical form, structure, biological properties, and development processes of the organism. With respect to the present disclosure, the phenotypes would refer to set of observable characteristics related to a disease. Herein, each of the phenotypes would be related to one or more phenotypic targets i.e., the target molecules or proteins having an association with the phenotypes. The disclosed system uses in-silico techniques i.e., the techniques which involve the role of databases and machine learning, for screening phenotypic targets associated with the disease over using in-vitro techniques, as the traditional in-vitro techniques are highly time consuming and resource intensive.

The system is further communicably coupled to a phenotype ontological databank comprising a plurality of phenotypes and phenotypic targets associated with each of the plurality of phenotypes. Herein, the phenotype ontological databank uses ontology, which is a data model that represents concepts, attributes, and relationships in the form of a directed acyclic graph. Furthermore, the phenotype ontological databank comprises a set of databases that contain information regarding phenotypes and the phenotypic targets related to each of the phenotypic targets. The phenotypic ontological bank plays a vital role in the in-silico screening of the phenotypic targets associated to the disease as all the data and knowledge regarding the phenotypes and the respective phenotypic targets of each of the phenotype is contained within the phenotype ontology databank. Optionally, the phenotype ontological databank comprises of a plurality of publicly available databases. Databases such as QuickGo®, Gene Ontology®, Human phenotype ontology, Monarch Initiative are some examples of publicly available databases that can be a part of the phenotype ontological databank. Moreover, the phenotype ontological databank is communicably coupled to the system in order to facilitate exchange of data between the phenotype ontological databank and system for the in-silico screening of the phenotypic targets associated with the disease.

Throughout the present disclosure, the term “processor” refers to a computational element that is operable to respond to and process instructions that drive the system. Furthermore, the processor may refer to one or more individual processors, processing devices and various elements associated with a processing device that may be shared by other processing devices. Additionally, the one or more individual processors, processing devices and elements are arranged in various architectures for responding to and processing the instructions that drive the system.

Throughout the present disclosure, the term “memory” refers to a volatile or persistent medium, such as an electrical circuit, magnetic disk, virtual memory or optical disk, in which a computer can store data or software for any duration. Optionally, the memory is a non-volatile mass storage such as physical storage media. Furthermore, a single memory may encompass and, in a scenario, wherein the system is distributed, the processing, memory and/or storage capability may be distributed as well.

The system comprises a processor communicably coupled to a memory, wherein the processor is configured to receive a first input of the disease. Herein, the first input corresponds to a form of information associated to the disease such as the name of disease, received by the processor to clearly indicate to the system about the specific disease for which the phenotypic targets are to be screened. For example, the name of the disease “Pancreatic Cancer” is received by the processor as the first input of the disease, to indicate to the system to screen for the phenotypic targets associated to pancreatic cancer. Optionally, the first input of the disease received by the processor is selected from a list of diseases. In essence, a disease of interest is selected from within the list of diseases, which is subsequently received by the processor as the first input of the disease. Furthermore, the selection of the first input of the disease from within the list may either be automated or performed by a user. Optionally, in case the selection is done by the user, then a user-interface may be provided to the user over a platform through which the user can select the first input of the disease from a list of diseases. Herein, the user refers to any person who wants to use the system for screening of phenotypic targets for the disease of their choice. Additionally, the platform can be in the form of a website or application that allows the user to access the system, wherein the user-interface is a point at which the user interacts with the platform to access the system.

In an embodiment, the first input of the disease may be “Pancreatic Cancer”. Subsequently, a list of cellular phenotypes and molecular phenotypes is procured for the first input. Thereafter, the list of cellular phenotypes and the molecular phenotypes are evaluated for genes, and perturbation value (p-value) is determined. Herein, the p-value provides probability of overlap seen between target of a drug and gene target to be significant. Typically, lower the p-value, more significant is the overlap. The structure is as shown in Table 1

TABLE 1

| Phenotypes | Genes | p-value |
|---|---|---|
| double-strand break | BRCA2 \| RAD51D \| | 6.38E-12 |
| repair via homologous | RAD51 \| FAN1 \| | |
| recombination | MRE11 \| PALB2 \| | |
| | BRCA | |
| single-stranded DNA | BRCA2 \| RAD51D \| | 1.69E-08 |
| binding | RAD51 \| MLH1 \| | |
| | PMS2 \| MSH2 | |
| DNA repair | MSH6 \| RAD50 \| | 2.37E-08 |
| | FAN1 \| NPM1 \| | |
| | MSH2 \| RAD51C | |
| telomerase RNA | TERT \| WRAP53 \| | 8.34E-10 |
| binding | NOP10 \| NHP2 \| | |
| | DKC1 | |
| negative regulation of | CDKN1B \| NPM1 \| | 2.73E-10 |
| cell population | CDKN2A \| MEN1 \| | |
| proliferation | WT1 \| APC \| | |
| | STK11 \| | |
| negative regulation of | CDKN1B \| MEN1 \| | 1.71E-09 |
| cyclin-dependent | CDKN2A \| APC \| | |
| protein serine/ | PTEN | |
| threonine | | |
| positive regulation of | POT1 \| ACD \| | 3.79E-08 |
| telomerase activity | DKC1 \| PARN \| | |
| | WRAP53 | |
| negative regulation of | CDKN1B \| CDKN2A \| | 6.99E-06 |
| cell growth | WT1 \| TP53 \| | |
| | SMAD4 | |
| mismatch repair | MSH6 \| MSH2 \| | 1.26E-07 |
| | MLH1 \| PMS2 | |
| double-stranded DNA | MSH2 \| RAD51 \| | 1.26E-05 |
| binding | MSH6 \| MENI | |
| cellular response to | RAD51 \| RAD50 \| | 1.48E-08 |
| DNA damage stimulus | MRE11 \| MEN1 \| | |
| | APC \| STK11 \| | |
| | CCND1 \| | |
| ATP binding | RTEL1 \| RAD51 \| | 2.07E-05 |
| | MSH6 \| STK11 \| | |
| | MSH2 \| BMPRIA | |
| negative regulation of | POT1 \| CTC1 \| | 3.53E-07 |
| telomere maintenance | ACD \| TINF2 | |
| via telomerase | | |
| telomeric DNA binding | POT1 \| CTC1 \| | 5.42E-07 |
| | ACD \| TINF2 | |
| positive regulation of | TP53 \| BARD1 \| | 0.01484161162 |
| apoptotic process | APC | |
| positive regulation of | CDK4 \| NPM1 \| | 0.001594139122 |
| cell population | EPCAM \| KRAS \| | |
| proliferation | PDGFRB | |
| protein localization | TP53 NPM1 | 0.004019637206 |
| protein-containing | CDKNIB \| ACD \| | 0.000106731638 |
| complex binding | EPCAM \| KRAS | |
| | WRAP53 | |

The processor is then configured to receive a second input relating to at least one phenotype associated with the disease. Herein, upon receiving the first input of the disease, the processor receives a second input where the second input comprises of one or more than one phenotype having some association with the disease of interest for which the phenotypic targets are to be screened. Optionally, the second input relating to at least one phenotype associated with the disease is in the form of a cellular, molecular or clinical phenotype. Herein, the cellular phenotype corresponds to a cellular process that involves gene and protein expression. Furthermore, the molecular phenotype corresponds to the disease affecting a molecule at molecular levels directly. Additionally, the clinical phenotype corresponds to the clinical symptoms caused due to the disease. For instance, the processor may receive the second input, wherein the second input may be for example, “pancreatic stellate cell proliferation” or “abnormality of exocrine pancreas physiology” or both of them. Herein, the second input relates to at least one phenotype associated with the disease “Pancreatic Cancer”, wherein the disease “Pancreatic Cancer” is received by the processor as the first input.

The processor is then configured to identify for each of the at least one phenotype a plurality of similar phenotypes relating to a particular phenotype of the at least one phenotype of the second input. In essence, the second input received by the processor, relates to one or more phenotype associated with the disease. Subsequently, the processor uses the phenotype ontological databank which is communicably coupled to the system, to separately identify the similar phenotypes to the particular phenotype of the at least one phenotype from among the data of all the phenotypes present in the databank for each of the phenotype of the at least one phenotype. Thereby, the information of the identified similar phenotypes is then passed on to the system. Herein, the plurality of phenotypes refers to the phenotypes that are similar to the particular phenotype of the at least one phenotype received as the second input. In order to identify the similar phenotypes from the phenotype ontological databank, the processor uses literature mining. For example, in case the second input received by the processor are two phenotypes associated with the disease as, then the processor would separately identify similar phenotypes for each of the two phenotypes received by the system as the second input. One or more similarity algorithms can be employed to obtain comprehensive similarity. In an implementation, the similarity algorithm is a Euclidean distance algorithm. In another implementation, the similarity algorithm is Random Walk with Restart (RWR) algorithm-based method.

The processor is then configured to determine a similarity score for each of the plurality of similar phenotypes in comparison with the particular phenotype of the at least one phenotype of the second input. Herein, the similarity score will be a numerical value that will represent the similarity of the identified phenotype in comparison to the particular phenotype of the at least one phenotype. For example, the similarity score of the identified phenotype can be a number between ‘0’ to ‘1’, wherein the similarity score closer to ‘1’ represents that the identified phenotype is more similar to the particular phenotype of the at least one phenotype and vice versa. In a similar way, the processor determines the similarity score for each of the phenotype in the plurality of similar phenotypes which will represent the similarity of the respective phenotype in comparison to the particular phenotype of the at least one phenotype of the second input. The plurality of the similar phenotypes can be further arranged into a form of a list prioritized on the basis of the similarity score.

In an embodiment, the processor is configured to identify for each of the at least one phenotype a plurality of similar phenotypes for the input “Pancreatic cancer”, related to the particular phenotype of the at least one phenotype of the second input. Furthermore, the processor is configured to determine the similarity score for each of the plurality of similar phenotypes in as shown in Table 2

TABLE 2

| Phenotypes for which Phenotypic | |
|---|---|
| Targets will be fetched | Similarity score |
| double-strand break repair via | 0.6407105002 |
| homologous recombination | |
| DNA repair | 0.505385618 |
| negative regulation of cell | 0.5026048842 |
| population proliferation | |
| negative regulation of cyclin- | 0.4937651134 |
| dependent protein | |
| serine/threonine | |
| positive regulation of telomerase | 0.4528600852 |
| activity | |
| negative regulation of cell growth | 0.4363899122 |
| mismatch repair | 0.4276367718 |
| cellular response to DNA damage | 0.4107372361 |
| stimulus | |
| negative regulation of telomere | 0.3909633151 |
| maintenance via telomerase | |
| positive regulation of apoptotic | 0.3677218124 |
| process | |

The processor is further configured to extract, from the phenotype ontological databank, phenotypic targets associated with similar phenotypes having similarity score higher than a first predefined threshold. Herein, the processor from within the plurality of similar phenotypes, looks for similar phenotypes that comprises the similarity score that is higher than the first predefined threshold. Herein, the first predefined threshold may be a numerical value that is set to a default value in the system or may be by the user according to requirements of the user. For example, the first predefined threshold is by the user, wherein the first predefined threshold may be ‘0.75’. In this scenario, the processor looks out for similar phenotypes having the similarity score higher than ‘0.75’ from within the plurality of similar phenotypes. Subsequently, after identifying the similar phenotypes having the similarity score higher than the first predefined threshold, the processor extracts the phenotypic targets associated with the respective similar phenotype for each of such similar phenotype with the similarity score higher than the first predefined threshold. The phenotypic targets are extracted by the processor from the phenotype ontological databank which is communicably coupled to the system using literature mining. In an example, the similar phenotypes having the similarity score higher than ‘0.5’ within the plurality of similar phenotypes are extracted for the phenotype “pancreatic stellate cell proliferation” as shown in the structure as shown in Table 3

TABLE 3

| Phenotypes | Similarity score |
|---|---|
| Pancreatic stellate cell proliferation | 1 |
| Negative regulation of pancreatic stellate cell | 0.833 |
| proliferation | |
| Positive regulation of pancreatic stellate cell | 0.833 |
| proliferation | |
| Regulation of pancreatic stellate cell | 0.833 |
| proliferation | |
| Fibroblast proliferation | 0.8 |
| Regulation of fibroblast proliferation | 0.667 |
| Hepatic stellate cell proliferation | 0.667 |
| Positive regulation of fibroblast proliferation | 0.667 |
| Negative regulation of fibroblast proliferation | 0.667 |
| Fibroblast proliferation involved in heart | 0.667 |
| morphogenesis | |
| Cell population proliferation | 0.6 |
| Negative regulation of hepatic stellate cell | 0.571 |
| proliferation | |
| Positive regulation of hepatic stellate cell | 0.571 |
| proliferation | |
| Regulation of hepatic stellate cell proliferation | 0.571 |

The processor is further configured to compute a cumulative score of the phenotypic targets based on a plurality of parameters, wherein the cumulative score of a given phenotypic target is indicative of relevance thereof with respect to the disease. Herein, the cumulative score is a numeric score that is computed based on the plurality of parameters i.e., based on more than one parameter and the individual score of each of the parameter is added to compute the final cumulative score. Furthermore, each of the given phenotypic target are assigned the cumulative score computed by the processor based on the plurality of parameters. Herein, the phenotypic target comprising a higher cumulative score indicates that the phenotypic target is of more relevance to the disease.

Optionally, the plurality of parameters to compute the cumulative score of the phenotypic targets comprise
whether the phenotypic target is modified post transitionally, wherein, the phenotypic target may indicate differential post translational modification (PTM) in the disease by acting as a substrate or by mediating PTM of other protein by acting as an enzyme, such as, kinase, phosphatase, in the disease. In case, the phenotypic target is either undergoing the PTM or mediating the PTM of some other protein in the disease specifically, then a higher score is generated for the phenotypic target for the specific parameter,
whether the phenotypic target is differentially expressed i.e., in case the phenotypic target undergoes differential gene expression by action of the disease, then depending upon magnitude of change in expression of the differential gene expression (represented as fold change), a score is given. Herein, higher the change in differential expression, wherein the differential expression may be upregulated or downregulated, higher is the scoring. Furthermore, data from databases comprising differentially expressed genes are used to obtain information about gene expression changes in the disease of the phenotypic target. In particular, phenotypic targets which are not differentially expressed, do not get any score.
whether the phenotypic target is modulated by differentially expressed microRNA (miRNA) i.e., herein if the miRNAs regulate the expression of the phenotypic target, then the score is generated for the phenotypic target,
whether the phenotypic target is modulated by differentially expressed non-coding RNA (ncRNA), wherein the ncRNAs regulate the expression of the phenotypic target is collected. Thereafter, in case the ncRNAs regulate expression of genes of the phenotypic targets, then a score is given. However, in case the phenotypic target is not regulated by differentially expressed ncRNAs, then the score is zero,
the phenotypic target's single nucleotide polymorphisms (SNP) and association with the disease i.e., herein, the phenotypic target is given the score based upon the number of pathways formed in association with the disease,
the expression quantitative trait loci (eQTL) and Allelic-fold change (AFC) score in the tissue which is most implicated in the disease, wherein values of eQTL provide information regarding influence of genetic polymorphisms in a gene on expression phenotype at population level with respect to phenotypic target,
the co-occurrence score from publications, grants, patents, clinical trials, congresses, media between the phenotypic target and the disease i.e., herein, the data-mining approach is used to determine co-occurrence between the phenotype target and the disease, and is searched in different asset classes, publications, grants, patents, clinical trials, thesis, media reports and score is given based on the co-occurrence.

Each of the phenotypic target would be given the individual score for each of the parameter and then the individual scores are added up to compute the cumulative score of each of the phenotypic target.

In an embodiment, the plurality of parameters to compute the cumulative score of the phenotypic targets may comprise a score that indicates genetic variation of target with respect to the disease, wherein genetic variations in phenotypic targets of the disease comprises count and frequency, which are used to generate a score. Herein, a higher count and frequency of genetic variations contribute to a higher score. However, phenotypic targets without any genetic link to the disease do not get any score. Furthermore, the plurality of parameters to compute the cumulative score of the phenotypic targets may comprise a score that indicates expression of phenotypic target in the tissue that is tissue most relevant for pathology of the disease provided as the input. Herein, known expression levels of the phenotypic target are dependent upon ribonucleic acid (RNA) and protein level in the tissue where disease phenotypes are observed. Additionally, a higher expression of the phenotypic targets corresponds to a higher score.

In an embodiment, the genetic variation of the phenotypic targets may be for example, “TP53”, wherein “TP53” comprises the cumulative score of the phenotypic targets, and a list of drugs related to the genetic variation of the phenotypic targets as shown in Table 4

TABLE 4

| Genetic variation of | | |
|---|---|---|
| the phenotypic | | |
| targets | Cumulative score | List of drugs |
| TP53 | 43.705 | H 101 \| ALRN 6924 \| |
| | | contusugene ladenovec \| |
| | | Acetylsalicylic acid \| |
| | | Lesogaberan \| |
| | | Thioureidobutyronitrile \| |
| | | 1-(9-ethyl-9H-carbazol-3- |
| | | yl)-N-methylmethanamine \| |
| | | p28 Peptide \| COTI 2 \| |
| | | Cenersen \| CX 5461 \| MVA |
| | | p53 vaccine \| APR-246 \| |
| | | SCH 58500 \| CGM 097 \| |
| | | SGT 94 \| CBLC 137 \| CXS |
| | | 299 \| H 103 \| bacitracin |
| | | zinc, polymyxin b sulfate \| |
| | | Triethyl Phosphate \| SGT |
| | | 53 \| Pifithrin-alpha \| SL |
| | | 801 \| INGN 225 \| PRIMA-1 \| |
| | | MRX 34 \| SAR 405838 \| |
| | | AFP 464 \| Zinc gluconate \| |
| | | Zinc \| CYANOCOBALAMIN |
| KRAS | 38.410 | BOCEPREVIR \| Simeprevir \| |
| | | MRTX 849 \| faldaprevir \| |
| | | FARNESYL DIPHOSPHATE \| |
| | | [(3,7,11-TRIMETHYL- |
| | | DODECA-2,6,10- |
| | | TRIENYLOXYCARBAMOYL)- |
| | | METHYL]-PHOSPHONIC |
| | | ACID \| Paritaprevir \| |
| | | TELAPREVIR \| Lonafarnib \| |
| | | Sotorasib |
| PTEN | 35.030 | Phosphatidylethanolamine |
| BRCA2 | 32.751 | |
| EPCAM | 32.197 | Anti-idiotype colorectal |
| | | cancer vaccine \| |
| | | Tucotuzumab celmoleukin \| |
| | | Anti-KSA cancer vaccine \| |
| | | catumaxomab \| IGN 101 \| |
| | | Anti-17-1A monoclonal |
| | | antibody 3622W94 \| Tc |
| | | 99m nofetumomab |
| | | merpentan \| ING-1 \| |
| | | CIDOFOVIR \| Citatuzumab |
| | | bogatox \| Oportuzumab |
| | | monatox \| Adecatumumab \| |
| | | AMG 110 \| Monoclonal |
| | | antibody 323A3 \| VB 2011 \| |
| | | Hypromellose \| NRLU 10 |
| PIK3CA | 31.727 | Pilaralisib \| Pictrelisib \| LY |
| | | 3023414 \| Voxtalisib \| |
| | | AMG 319 \| Taselisib \| MEN |
| | | 1611 \| Wortmannin \| |
| | | Puquitinib \| WX 037 \| |
| | | CUDC 907 \| 1-cyclopentyl- |
| | | 3-(1H-pyrrolo[2,3- |
| | | b]pyridin-5-yl)-1H- |
| | | pyrazolo[3,4-d]pyrimidin- |
| | | 4-amine \| BAY 1082439 \| |
| | | Seletalisib \| Adenosine |
| BRIP1 | 29.788 | |
| PMS2 | 29.401 | Adenosine 5′-[y- |
| | | thio]triphosphate |
| BRCA1 | 29.000 | |
| BAP1 | 27.761 | |

The processor is then configured to screen phenotypic targets with cumulative score lower than a second predefined threshold. Herein, the second predefined threshold is a numeric value of the cumulative score that is either set by default in the system or is set by the user according to the requirements of the user. The processor proceeds to screen out those phenotypic targets that have the cumulative score lower than the second predefined threshold. Subsequently, the screened phenotypic targets are prioritized based on the cumulative score. Herein, the process of screening refers to separate identification of the phenotypic targets. For example, if the second predefined threshold is set at a value, that may be for example, ‘30’, then the processor screens out all the phenotypic targets having the cumulative score lower than the value ‘30’.

The processor is further configured to compute relevant pathways for the phenotypic targets by performing Highly dysregulated pathway analysis (HDPA) for the screened phenotypic targets. Herein, HDPA takes into account data about differential expressions of genes, to gain mechanistic insights into the phenotypic targets that are observed. Furthermore, HDPA comprises fold change (FC) values, that indicates magnitude of change in gene expression, wherein the change in gene expression may be upregulated or downregulated. Herein, the FC is a measure describing degree of quantity change between final relevant pathways of the phenotypic targets and original relevant pathways of the phenotypic targets. Additionally, FC values are used to perform quantitative analysis of impact on signaling pathways. Herein, the pathways which are most impacted get a highest perturbation (p-dys) score.

Optionally, the processor is configured to form Highly dysregulated pathway analysis (HDPA) using differential expression analysis of screened phenotypic targets. Herein, analysis of impact of the pathways is based on at least two types of data. Herein, firstly, the differentially expressed genes are over-represented in a given pathway as mentioned in the present disclosure. Secondly, abnormal perturbation of the relevant pathway is measured by propagating measured expression changes across pathway topology. Furthermore, the differentially expressed genes which are over-represented in a given pathway is denoted by an independent first probability “P_{NDE}” and the abnormal perturbation of the pathway is denoted by an independent second probability, “P_{PERT}”. Herein, the first probability captures the significance of a given pathway as provided by the over-representation analysis of the number of differentially expressed genes observed on the pathway. Furthermore, value of the “P_{NDE}” represents the probability of obtaining a number of differentially expressed genes on the given pathway at least as large as observed pathway. Herein, the first probability is

P_{NDE}=P(X≥N_{DE}|H_{O})

wherein, H_{O} denotes null hypothesis, wherein the genes that appear as differentially expressed on the given pathway is completely random, N_{DE} denotes number of differentially expressed genes on the pathway analyzed. Notably, the relevant pathways computed for the phenotypic targets using HDPA uses information regarding differentially expressed genes in control with respect to the disease condition only. Moreover, the second probability is calculated based on amount of perturbation measured in each pathway.

Optionally, the processor is configured to form a Pathway-Target-Phenotype (PTP) network using interactions between the screened phenotypic targets and most impacted pathways obtained from the results of HDPA. Herein, the first probability “P_{NDE}” and the second probability “P_{PERT}” are combined into one global probability value, denoted by “P_{G}”, that is used to rank the pathways and evaluate the perturbation of the pathway. Thereafter, the PTP network is formed using interactions between the screened phenotypic targets and most impacted pathways to find out disease pathways through analysis of the PTP network. Additionally, HDPA combines the differentially expressed gene expressions and information from structure of the pathway. Herein, effect of alteration of gene expression at different positions in the pathway is considered to be different.

The processor is further configured to compute mechanistic factors attributing to regulation of similar phenotypes and pathological information of the disease in association with the screened phenotypic targets. Herein, the mechanistic factors may be evaluated using an advanced network, wherein the advanced network constitutes entities such as the phenotypic targets, drug target genes and pathways on edges that indicate direction and direction types among the entities. Furthermore, the advanced network enables closest path between drug-phenotype or phenotypic target-phenotype, thereby highlighting and comparing important motifs that involve phenotypic targets-phenotypes-pathways.

Moreover, the present description also relates to the method as described above. The various embodiments and variants disclosed above apply mutatis mutandis to the present method.

Optionally, the method in the present disclosure wherein the first input of the disease received by the processor is selected from a list of diseases.

Optionally, the method in the present disclosure wherein the second input relating to at least one phenotype associated with the disease is in the form of a cellular, molecular or clinical phenotype.

Optionally, the method in the present disclosure wherein the phenotype ontological databank comprises of a plurality of publicly available databases.

Optionally, the method in the present disclosure wherein the plurality of parameters to compute the cumulative score of the phenotypic targets comprising:
whether the phenotypic target is modified post transitionally
whether the phenotypic target is differentially expressed
whether the phenotypic target is modulated by differentially expressed microRNA (mRNA)
whether the phenotypic target is modulated by differentially expressed non-coding RNA (ncRNA)
the phenotypic target's single nucleotide polymorphisms (SNP) and association with the disease
the expression quantitative trait loci (eQTL) and Allelic-fold change (AFC) score in the tissue which is most implicated in the disease
the co-occurrence score from the publications, grants, patents, clinical trials, congresses, media between the phenotypic target and the disease
the impact factor score between the phenotypic target and the disease.

Optionally, the method in the present disclosure wherein the processor is configured to perform Highly dysregulated pathway analysis (HDPA) using differential expression analysis of screened phenotypic targets.

Optionally, the method in the present disclosure wherein the processor is configured to form a Pathway-Target-Phenotype (PTP) network using interactions between the screened phenotypic targets and most impacted pathways obtained from the results of HDPA.

The system and the method of the present disclosure may be employed for more extensively using the phenotypes and the phenotypic targets in studying the pathology of diseases. Further, the disclosed system and method does not rely upon evaluating the role of just a single phenotype and hence, consider the role of multiple phenotypes and phenotypic targets simultaneously associated to the disease to gain further insights into the pathology of the disease.

### DETAILED DESCRIPTION OF DRAWINGS

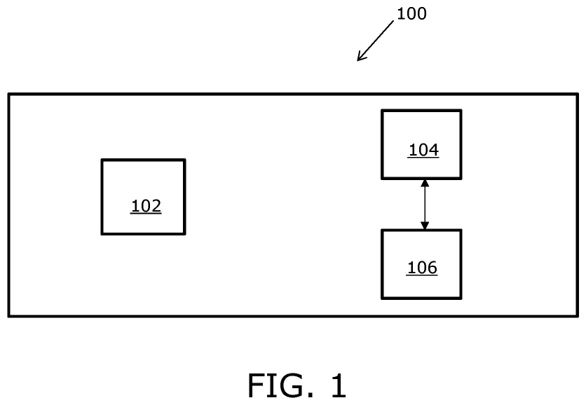

Referring to FIG. 1, there is shown a block diagram of a system 100 for screening phenotypic targets associated with a disease using in-silico techniques, in accordance with the embodiments of the present disclosure. Herein, the system 100 comprises a phenotype ontological databank 102, wherein the phenotype ontological databank 102 comprises a plurality of phenotypes and phenotypic targets corresponding to each of the plurality of drugs thereof. Furthermore, the system 100 comprises a processor 104 communicably coupled to a memory 106.

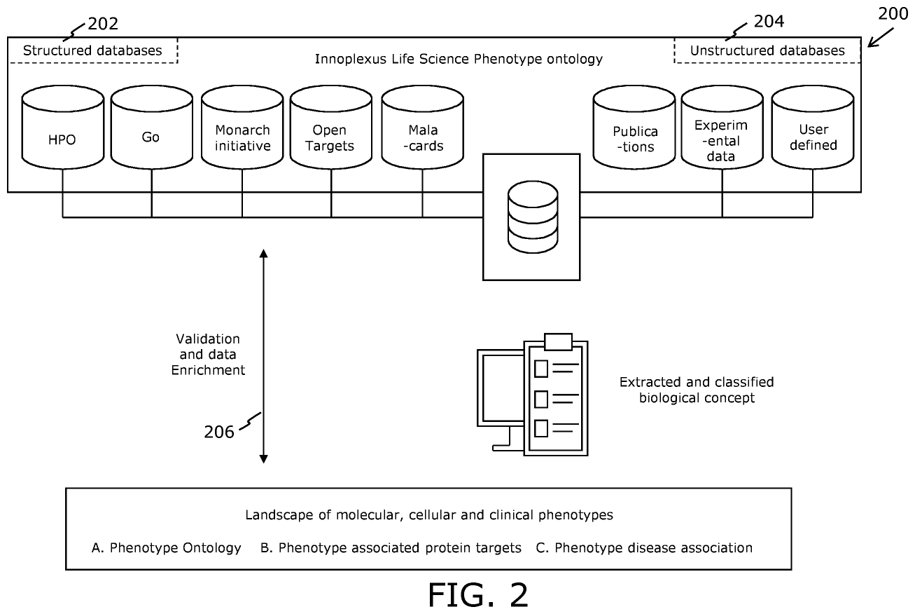

Referring to FIG. 2, there is shown a system 200 for determining phenotypic targets of at least one drug, in accordance with the implementation of the present disclosure. Herein, structured databases 202 such as Human Phenotype Ontology (HPO), Gene Ontology (GO), Monarch Initiative, and so forth may be used along with unstructured databases 204 such as publications, experimental data and/or user defined terminology. Subsequently, biological concepts are extracted and classified in the form of a landscape of molecular phenotype, cellular phenotype and clinical phenotypes, thereby deriving phenotype ontology, phenotype associated protein targets and phenotype disease association. The structured databases are communicably coupled 206 with the landscape of molecular phenotype, cellular phenotype and clinical phenotypes for validation and data enrichment.

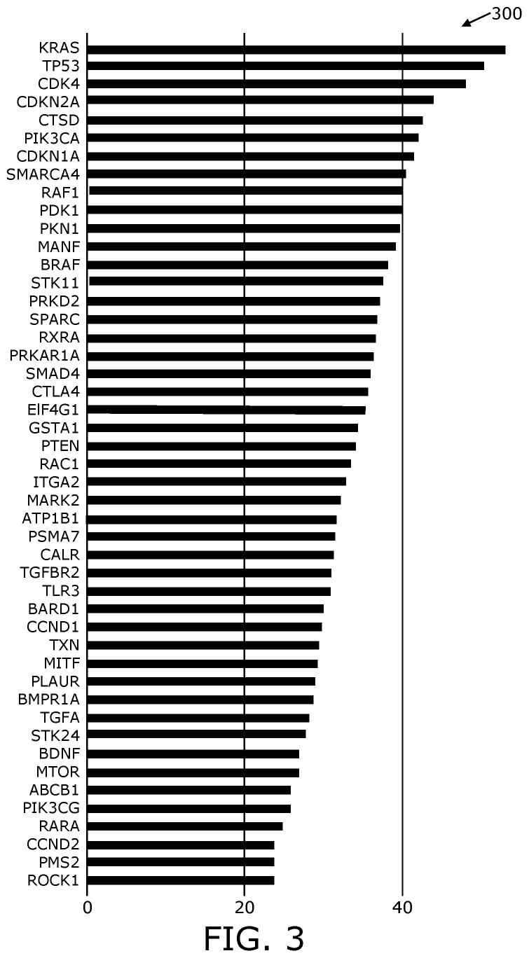

Referring to FIG. 3, there is shown a graph 300 to prioritize the screened phenotypic targets based on the cumulative score, in accordance with implementation of the present disclosure. Herein, the horizontal axis represents the cumulative score of the screened phenotypic target. The vertical axis represents the various screened phenotypic targets.

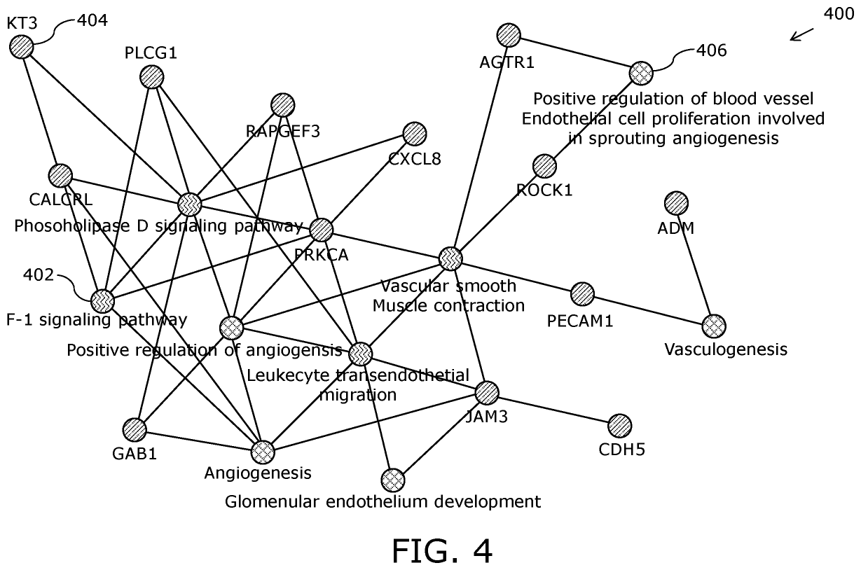

Referring to FIG. 4, there is shown a Pathway-Target-Phenotype network (PTP) 400, in accordance with the embodiments of the present disclosure. Herein, the PTP network 400 comprises direct and indirect relation to at least one pathway 402 with phenotypic targets 404 and phenotypes 406 in association with the disease. Furthermore, the PTP network 400 are visually represented as simple graphs, with nodes and vertices, wherein the nodes have a number of edges attached to it. Herein, the pathway is denoted by “P”, the phenotypic target by “T” and the phenotype by “P”.

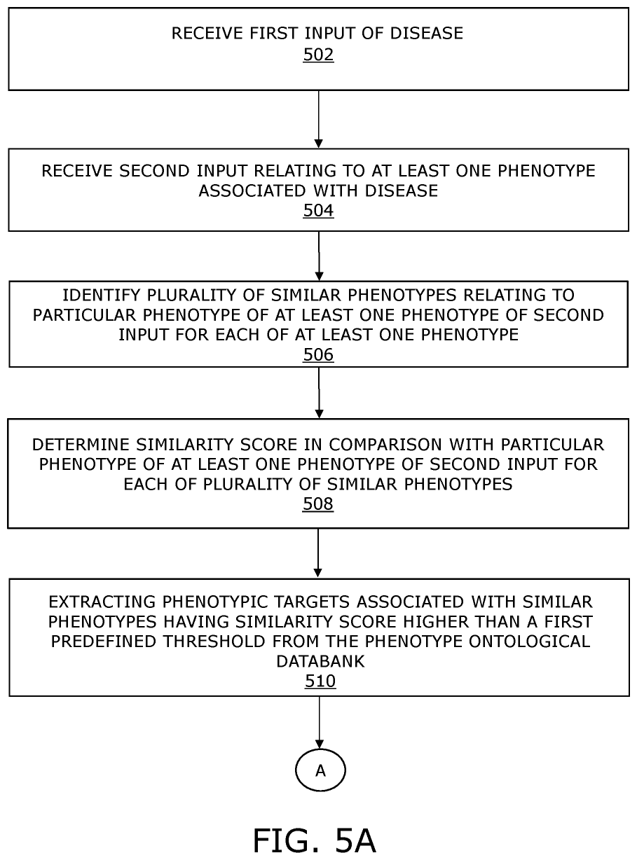

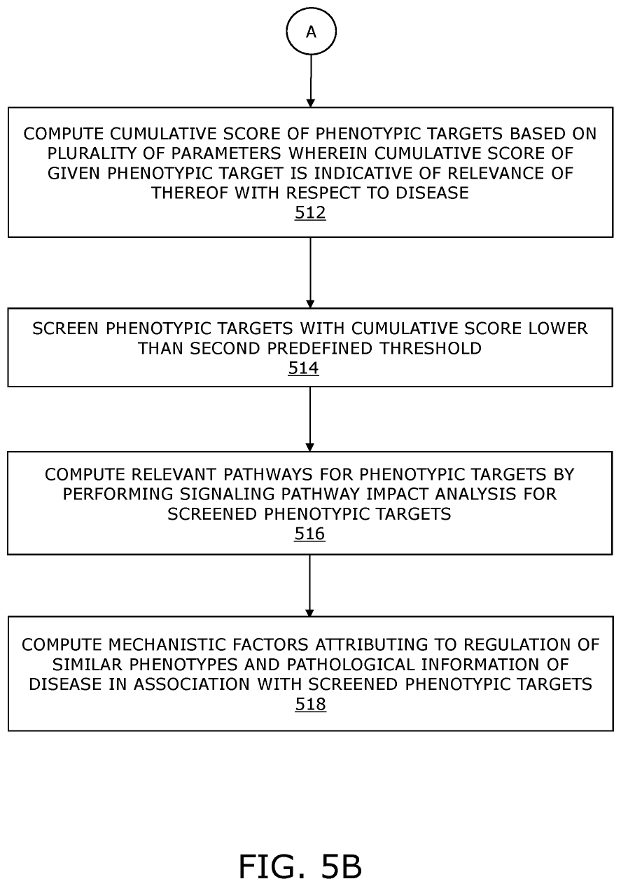

Referring to FIGS. 5A and 5B collectively, there is shown a flowchart depicting steps of a method for screening phenotypic targets associated with a disease using in-silico techniques, in accordance with the embodiments of the present disclosure. At step 502, a first input of the disease is received. At step 504, a second input relating to at least one phenotype associated with the disease is received. At step 506, a plurality of similar phenotypes relating to the particular phenotype of the at least one phenotype of the second input for each of the at least one phenotype is identified. At step 508, a similarity score in comparison with the particular phenotype of the at least one phenotype of the second input for each of the plurality of similar phenotypes is determined. At step 510, phenotypic targets associated with similar phenotypes having similarity score higher than a first predefined threshold are extracted from the phenotype ontological databank. At step 512, a cumulative score of the phenotypic targets based on a plurality of parameters wherein the cumulative score of a given phenotypic target is indicative of relevance of thereof with respect to the disease is computed. At step 514, phenotypic targets with cumulative score lower than a second predefined threshold are screened. At step 516, relevant pathways for the phenotypic targets by performing Highly dysregulated pathway analysis (HDPA) for the screened phenotypic targets are computed. At step 518, mechanistic factors attributing to regulation of similar phenotypes and pathological information of the disease in association with screened phenotypic targets are computed.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A system for screening phenotypic targets associated with a disease using in-silico techniques, the system communicably coupled to
a phenotype ontological databank comprising information pertaining to a plurality of phenotypes and phenotypic targets associated with each of the plurality of phenotypes;
wherein the system comprises a processor communicably coupled to a memory, and wherein the processor is configured to execute machine readable instructions that cause the system to perform the following operation:
receive a name of the disease as a first input;
receive at least one phenotype associated with the disease as a second input;
identify for each of the at least one phenotype a plurality of similar phenotypes relating to a particular phenotype of the at least one phenotype of the second input;
determine a similarity score for each of the plurality of similar phenotypes in comparison with the particular phenotype of the at least one phenotype of the second input;
extract, from the phenotype ontological databank, phenotypic targets associated with similar phenotypes having similarity score higher than a first predefined threshold;
compute a cumulative score of the phenotypic targets based on a plurality of parameters, wherein the cumulative score of a given phenotypic target is indicative of relevance thereof with respect to the disease;
screen out phenotypic targets with cumulative score lower than a second predefined threshold;
compute relevant pathways for the phenotypic targets by performing Highly dysregulated pathway analysis (HDPA) for the screened phenotypic targets;
compute mechanistic factors attributing to regulation of similar phenotypes and pathological information of the disease in association with the screened phenotypic targets.

2. A system according to claim 1 wherein the first input of the name of the disease received by the processor is selected from a list of diseases.

3. A system according to claim 1 wherein the second input relating to at least one phenotype associated with the disease is in the form of a cellular, molecular or clinical phenotype.

4. A system according to claim 1 wherein the phenotype ontological databank comprises of a plurality of publicly available databases.

5. A system according to claim 1 wherein the plurality of parameters to compute the cumulative score of the phenotypic targets comprising:
whether the phenotypic target is modified post transitionally;
whether the phenotypic target is differentially expressed;
whether the phenotypic target is modulated by differentially expressed microRNA (miRNA);
whether the phenotypic target is modulated by differentially expressed non-coding RNA (ncRNA);
the phenotypic target's single nucleotide polymorphisms (SNP) and association with the disease.
the expression quantitative trait loci (eQTL) and Allelic-fold change (AFC) score in the tissue which is most implicated in the disease;
the co-occurrence score from the publications, grants, patents, clinical trials, congresses, media between the phenotypic target and the disease.

6. A system according to claim 1 wherein the processor is configured to perform Highly dysregulated pathway analysis (HDPA) using differential expression analysis of screened phenotypic targets.

7. A system according to claim 1 wherein the processor is configured to form a Pathway-Target-Phenotype (PTP) network using interactions between the screened phenotypic targets and most impacted pathways obtained from the results of HDPA.

8. A method for screening phenotypic targets associated with a disease using in-silico techniques, wherein the method is implemented using a system communicably coupled to
a phenotype ontological databank comprising information pertaining to a plurality of phenotypes and phenotypic targets associated with each of the plurality of phenotypes;
wherein the system comprises a processor communicably coupled to a memory, the method comprising:
receiving a name of the disease as a first input;
receiving at least one phenotype associated with the disease as a second input;
identifying for each of the at least one phenotype a plurality of similar phenotypes relating to the particular phenotype of the at least one phenotype of the second input;
determining a similarity score for each of the plurality of similar phenotypes in comparison with the particular phenotype of the at least one phenotype of the second input;
extracting, from the phenotype ontological databank, phenotypic targets associated with similar phenotypes having similarity score higher than a first predefined threshold;
computing a cumulative score of the phenotypic targets based on a plurality of parameters, wherein the cumulative score of a given phenotypic target is indicative of relevance thereof with respect to the disease;
screen out phenotypic targets with cumulative score lower than a second predefined threshold;
computing relevant pathways for the phenotypic targets by performing Highly dysregulated pathway analysis (HDPA) for the screened phenotypic targets;
computing mechanistic factors attributing to regulation of similar phenotypes and pathological information of the disease in association with the screened phenotypic targets.

9. A method according to claim 8 wherein the first input of the disease received by the processor is selected from a list of diseases.

10. A method according to claim 1 wherein the second input relating to at least one phenotype associated with the disease is in the form of a cellular, molecular or clinical phenotype.

11. A method according to claim 1 wherein the phenotype ontological databank comprises of a plurality of publicly available databases.

12. A method according to claim 1 wherein the plurality of parameters to compute the cumulative score of the phenotypic targets comprising:
whether the phenotypic target is modified post transitionally;
whether the phenotypic target is differentially expressed;
whether the phenotypic target is modulated by differentially expressed microRNA (miRNA);
whether the phenotypic target is modulated by differentially expressed non-coding RNA (ncRNA);
the phenotypic target's single nucleotide polymorphisms (SNP) and association with the disease;
the expression quantitative trait loci (eQTL) and Allelic-fold change (AFC) score in the tissue which is most implicated in the disease;
the co-occurrence score from the publications, grants, patents, clinical trials, congresses, media between the phenotypic target and the disease.

13. A method according to claim 1 wherein the processor is configured to perform Highly dysregulated pathway analysis (HDPA) using differential expression analysis of screened phenotypic targets.

14. A method according to claim 1 wherein the processor is configured to form a Pathway-Target-Phenotype (PTP) network using interactions between the screened phenotypic targets and most impacted pathways obtained from the results of HDPA.
